(19) United States
(12) Patent Application Publication
(10) Pub. No.: US 2025/0356835 A1
(43) Pub. Date: Nov. 20, 2025

(54) INTELLIGENT SYNTHESIS METHOD AND SYSTEM FOR CANTONESE SPEECH BASED ON ELECTROENCEPHALOGRAM EMOTION MEASUREMENT

(71) Applicants: Guangzhou University, Guangzhou (CN); Guangzhou Broadcasting Network, Guangzhou (CN)

(72) Inventors: Yongjun HU, Guangzhou (CN); Xianwu LIANG, Guangzhou (CN); Jianxin TENG, Guangzhou (CN); Qinshan LIU, Guangzhou (CN); Liuqian ZHU, Guangzhou (CN); Jiefeng HE, Guangzhou (CN); Mengmeng JIANG, Guangzhou (CN); Hao LIN, Guangzhou (CN); Yifan WAN, Guangzhou (CN)

(73) Assignees: Guangzhou University, Guangzhou (CN); Guangzhou Broadcasting Network, Guangzhou (CN)

(21) Appl. No.: 19/206,114

(22) Filed: May 13, 2025

(30) Foreign Application Priority Data
May 15, 2024 (CN) 202410603270.5

(51) Int. Cl. G10L 13/02; G06N 3/0464; G06N 3/09; G10L 25/30; G10L 25/63

(52) U.S. Cl. CPC G10L 13/02; G06N 3/0464; G06N 3/09; G10L 25/30; G10L 25/63

(57) ABSTRACT

An intelligent synthesis method and system for Cantonese speech based on electroencephalogram emotion measurement relates to the technical field of intelligent speech synthesis. The intelligent synthesis method includes: S1. acquiring data; S2. labeling data; S3. preprocessing data; S4. training an electroencephalogram emotion measurement model; S5. training an emotional speech synthesis model; and S6. performing speech synthesis. The intelligent synthesis method and system proposes an electroencephalogram emotion measurement model and an emotional speech synthesis model. The emotional speech synthesis model converts texts in a script into speeches, an audience listens to synthesized speeches when wearing a non-invasive electroencephalogram device, an electroencephalogram is generated, and the electroencephalogram generates an emotion measurement through the electroencephalogram emotion measurement model, which is conducive to optimizing speech generation under emotion measurement results and synthesizing emotionally rich speech that meets the empathy requirements of the audiences.

### CROSS REFERENCE TO THE RELATED APPLICATIONS

This application is based upon and claims priority to Chinese Patent Application No. 202410603270.5, filed on May 15, 2024, the entire contents of which are incorporated herein by reference.

### TECHNICAL FIELD

The present invention relates to the technical field of intelligent speech synthesis, and in particular, to an intelligent synthesis method and system for Cantonese speech based on electroencephalogram emotion measurement.

### BACKGROUND

Speech synthesis refers to automatic generation of speeches from texts using a computer. In the movie and television show industry, an automatic dubbing program automatically generates speeches from elements such as text lines, emotional expressions and timbre in a script, and then matches speeches to pictures, so that dubbing cost is greatly reduced. However, since the production of the movie and television shows needs to achieve an empathetic effect, the generated speeches have extremely high requirements on emotions, and therefore, the emotional factors have become the focus of research on speech synthesis of the movie and television shows.

Intelligent speech synthesis has made great progress thanks to the development of deep neural networks. The speech synthesis based on a deep neural network generally achieves the conversion from texts to speeches by constructing a parameterized neural network and using sample data in a text-to-speech pair form to model a mapping relationship between the texts and the speech. This simple data-driven speech synthesis method lacks mining and modeling of finer-grained factors such as timbre and emotion.

A general approach to emotional speech synthesis is to enable a model to learn emotional styles of speeches by explicit labels, that is, by manually labeling speeches with text labels representing emotions, the model learns rhythms of the speeches during the speech synthesis process, thereby achieving the effect of emotional speech generation. However, explicit text labels only enable the model to learn the average style of sample data and the basic rhythm of the speeches, and still lack fine-grained analysis of emotional rhythm. Moreover, this method of labeling speeches with text labels is highly dependent on a mode of modeling emotion measurement by a model designer and a subjective will. The effect of emotional style learning and expression needs to be improved.

The existing emotion measurement modeling is achieved by a discrete method and a continuous method. The mainstream discrete method includes several basic emotional states and related extensions, such as “anger”, “expectation”, “fear”, “sadness”, “trust”, “surprise”, and “joy”. In addition, there is a method based on a color palette theory that may further create other emotions using the basic emotional state as a primary color, as well as an emotion wheel representation method and attribute-based or hierarchical emotion quantization methods.

Compared with the discrete method for emotion measurement, the continuous method may represent a more detailed emotional state and has higher accuracy. The continuous method generally uses several basic coordinate axes to represent emotions. A commonly used method is a Valence-Arousal bipolar emotional quadrant system, which describes emotions from both Valence and Arousal dimensions.

Studies have shown that changes in a potential of a cerebral cortex may represent a lot of information related to human cognition. When a person listens to an audio, the emotional characteristics may arouse imagination, so that the potential of the cerebral cortex is changed. Such potential changes may be represented by electroencephalograms (EEG), and finer-grained information implied in the EEG, such as information of emotional changes of a person in audio, may be extracted by a signal processing method.

In general, since different audiences have different emotional empathy points for film and television content, the speech synthesis method based on the previous explicit label training cannot provide differentiated expressions for specific audiences. Based on this problem, a more fine-grained emotion modeling method is needed to guide the optimization of speech synthesis effects.

Therefore, there is an urgent need for those skilled in the art to provide an intelligent synthesis method and system for Cantonese speech based on electroencephalogram emotion measurement to solve the limitations in the prior art.

### SUMMARY

In view of the above, the present invention provides an intelligent synthesis method and system for Cantonese speech based on electroencephalogram emotion measurement, which optimizes speech generation under the emotion measurement results by constructing an electroencephalogram emotion measurement model and an emotional speech synthesis model, and synthesizes emotionally rich speech that meets the empathy requirements of the audiences.

To achieve the above objective, the present invention adopts the following technical solutions.

An intelligent synthesis method for Cantonese speech based on electroencephalogram emotion measurement includes the following steps:
S1. data acquisition: acquiring electroencephalogram emotion data of a tester after hearing a speech segment by using an electroencephalogram signal acquisition device;
S2. data labeling: performing emotion labeling on the acquired electroencephalogram emotion data to obtain labeled emotion extremum group data;
S3. data preprocess: preprocessing the emotion extremum group data to obtain preprocessed emotion extremum group data;
S4. electroencephalogram emotion measurement model training: performing electroencephalogram emotion measurement model training on a convolutional neural network by the preprocessed emotion extremum group data to obtain a trained electroencephalogram emotion measurement model;
S5. emotional speech synthesis model training: outputting a recognition result of the trained electroencephalogram emotion measurement model as an input of a vits model, and thus performing emotional speech synthesis model training to obtain a trained emotional speech synthesis model; and
S6. speech synthesis: performing speech synthesis on a to-be-dubbed movie and television show by the trained emotional speech synthesis model, and outputting a final speech synthesis result.

According to the foregoing method, optionally, the acquiring electroencephalogram emotion data of a tester after hearing a speech segment by using an electroencephalogram signal acquisition device in the S1 is specifically as follows:
a tester wears an electroencephalogram acquisition helmet and calibrates positions of electrodes, a recorder and a data acquisition software are started in sequence, the tester completes calibration processes of opening eyes, closing eyes and clicking a mouse according to instructions of the data acquisition software, a computer calculates signal calibration time offset in the calibration process, the tester hears speech segments, and the electroencephalogram acquisition helmet acquires electroencephalogram data of the tester, and marks time for starting playing and ending playing of the speech segments in the electroencephalogram data.

According to the foregoing method, optionally, the performing emotion labeling on the acquired electroencephalogram emotion data to obtain labeled emotion extremum group data in the S2 is specifically as follows:
the electroencephalogram emotion data obtained in the S1 are subjected to feature processing, electroencephalogram emotion data capable of representing emotion extrema are screened out, and emotion polarity labeling is performed on the electroencephalogram emotion data.

According to the foregoing method, optionally, the emotion extremum group data are an extremum group a, an extremum group b and an extremum group c classifying six emotions according to bipolarity of emotions.

According to the foregoing method, optionally, noise removal pretreatment is performed on the emotion extremum group data in the S3 to obtain emotion extremum group data after noise removal.

According to the foregoing method, optionally, the performing electroencephalogram emotion measurement model training on a convolutional neural network by the preprocessed emotion extremum group data to obtain a trained electroencephalogram emotion measurement model in the S4 is specifically as follows:
S41. inputting a signal: the input signal has two sections, including anchor sample data anchor and real-time input electroencephalogram data input, and the anchor sample data is the emotion extremum group data obtained in S3 after noise removal;
S42. extracting a feature: the electroencephalogram emotion measurement model mainly captures a time period of emotion fluctuations, so that the emotion extremum group data after noise removal is framed by a window sliding method, and framed data is represented as follows:

$${S}^{input} = {S}_{1}^{input} ⊗ {S}_{2}^{input} ⊗ {S}_{3}^{input} ⊗ … ⊗ {S}_{N}^{input} , {S}_{n}^{input} ∈ {ℝ}^{C \times M} ;$$

$${S}^{anchor} = {S}_{1}^{anchor} ⊗ {S}_{2}^{anchor} ⊗ {S}_{3}^{anchor} ⊗ … ⊗ {S}_{N}^{anchor} , {S}_{n}^{anchor} ∈ {ℝ}^{C \times M}$$

wherein S^{anchor}∈ [symbol] ^{C×T}, S^{input}∈ [symbol] ^{C×T}, and C are a number of channels, T is a time length, [symbol] is a real number field, M is a window size, ⊕ is splicing operation, and N is a number of frames;
the feature extraction performed on each frame by the convolutional neural network is represented as follows:

$${O}_{n} ( i , j ) = {\sum }_{c} {\sum }_{m} {S}_{n} ( i + c , j + m ) K ( c , m )$$

wherein K is a two-dimensional convolution kernel, and the convolution is performed along a channel vertically and along a time axis horizontally, Sₙ is each frame of signal, Oₙ∈ [symbol] ^{1×D} is a feature map representing an output result of each frame of signal after convolution, D is a D-dimensional vector, i is a vertical position index of a feature map pixel, j is a horizontal position index of a feature map pixel, c is a vertical position index of the convolution kernel, and m is a horizontal position index of the convolution kernel;
S43. anchoring coherent noise removal module based on an attention mechanism: the attention mechanism is applied to eliminate features similar to the anchor data, features of the anchor sample data are taken as K, and features of the emotion extremum group data after noise removal are taken as Q and V, which are represented as:

$$K = [ {O}_{1}^{a n c h o r} ; {O}_{2}^{a n c h o r} ; {O}_{3}^{a n c h o r} ; … ; {O}_{N}^{a n c h o r} ] , K ∈ {ℝ}^{N \times D} ;$$

$$Q = V = [ {O}_{1}^{input} ; {O}_{2}^{input} ; {O}_{3}^{input} ; … ; {O}_{N}^{input} ] , Q , V ∈ {ℝ}^{N \times D} ;$$

a distance is represented as:

$$Distance ( K , Q ) = ( {d}_{ij} ) , {d}_{ij} = {‖ {O}_{i}^{a n c h o r} - {O}_{j}^{input} ‖}_{2} ;$$

a distance from the attention mechanism is represented as:

$${A}^{input} = Attention ( Q , K , V ) = softmax ( Distance ( K , Q ) ) V , {A}^{input} ∈ {ℝ}^{N \times D}$$

wherein dᵢⱼ is a distance between an i^{th} feature and a j^{th} feature;
S44. classifying by a classifier: after a feature of A^{input} is compressed by a multi-layer convolutional neural network, the compressed feature is input into a fully connected neural network for emotion measurement prediction:

$$Z = convolution ( {A}^{i nput} ) , Z ∈ {ℝ}^{O}$$

$$\hat{e} = sigmod ( ANN ( Z ) ) , \hat{e} ∈ [ 0 , 1 ]$$

wherein Z is a flattened feature vector after convolution operation, a vector dimension is O, ANN is a fully connected neural network function, ê is a scalar value of 0-1 representing emotion measurement, the closer a predicted value is to 0, the more similar an input electroencephalogram signal input_trail is to an electroencephalogram signal anchor_trail for anchoring, otherwise, the more dissimilar, and an emotion measurement value of the input electroencephalogram signal is judged according to the scalar value; and
S45. training each sample labeled with the emotion measurement scale to obtain three trained electroencephalogram emotion measurement models

$$\{ {M}_{a}^{e e g} , {M}_{b}^{e e g} , {M}_{c}^{e e g} \} .$$

According to the foregoing method, optionally, the outputting a recognition result of the trained electroencephalogram emotion measurement model as an input of a vits model and thus performing emotional speech synthesis model training to obtain a trained emotional speech synthesis model in the S5 is specifically as follows:
the emotional speech synthesis model is divided into model learning speech reconstruction training and model emotion enhancement learning training;
S51. model learning speech reconstruction: performing model training by using the preprocessed emotion extremum group data obtained in the S3, learning speech reconstruction, and embedding emotion information in a text encoder in a vector form; wherein a loss function is a difference between a generated speech Mel spectrogram \hat{X}ₘₑₗ and a Mel spectrogram Xₘₑₗ of a real sample, that is, whether the speech is reconstructed, and a loss value for each sample is represented as:

$${loss}_{stage 1} = {loss}_{r e c o n} = {‖ {X}_{m e l} - {\hat{X}}_{m e l} ‖}_{1} ;$$

S52. model emotion enhancement learning: performing emotion recognition on Cantonese speech generated by artificial intelligence (AI) using the trained electroencephalogram emotion measurement model obtained in the S4, importing a feedback signal of a dubber on an AI dubbing emotion by a device to calibrate a coding loss function of an AI dubbing emotion coder, calculating a loss value, and completing model reinforcement learning training for the emotional speech synthesis model, wherein a total loss value calculation formula is represented as:

$${loss}_{stage 2} = {α loss}_{r e c o n} + {β loss}_{emotion}$$

$${loss}_{emotion} = - ( e log ( \hat{e} ) + ( 1 - e ) log ( 1 - \hat{e} ) )$$

wherein α and β are weight coefficients of reconstruction loss and emotion loss in a total loss of the model reinforcement learning.

An intelligent synthesis system for Cantonese speech based on electroencephalogram emotion measurement is applied to the intelligent synthesis method for Cantonese speech based on electroencephalogram emotion measurement according to any one of the foregoing aspects, and includes: a data acquisition module, a data labeling module, a data preprocessing module, an electroencephalogram emotion measurement model training module, an emotional speech synthesis model training module, and a speech synthesis module;
the data acquisition module is connected to an input end of the data labeling module, and is configured to acquire electroencephalogram emotion data of a tester after hearing a speech segment by using an electroencephalogram signal acquisition device;
the data labeling module is connected to an input end of the data preprocessing module, and is configured to perform emotion labeling on the acquired electroencephalogram emotion data to obtain labeled emotion extremum group data;
the data preprocessing module is connected to an input end of the electroencephalogram emotion measurement model training module, and is configured to preprocess the emotion extremum group data to obtain preprocessed emotion extremum group data;
the electroencephalogram emotion measurement model training module is connected to an input end of the emotional speech synthesis model training module, and is configured to perform electroencephalogram emotion measurement model training on a convolutional neural network by the preprocessed emotion extremum group data to obtain a trained electroencephalogram emotion measurement model;
the emotional speech synthesis model training module is connected to an input end of the speech synthesis module, and is configured to output a recognition result of the trained electroencephalogram emotion measurement model as an input of a vits model and thus perform emotional speech synthesis model training to obtain a trained emotional speech synthesis model; and
the speech synthesis module is configured to perform speech synthesis on a to-be-dubbed movie and television show by the trained emotional speech synthesis model and output a final speech synthesis result.

It may be seen from the foregoing technical solutions that, compared with the prior art, the present invention provides an intelligent synthesis method and system for Cantonese speech based on electroencephalogram emotion measurement, which has the following beneficial effects: the present invention proposes an electroencephalogram emotion measurement model and an emotional speech synthesis model, wherein the emotional speech synthesis model converts texts in a script into speeches, an audience listens to synthesized speeches when wearing a non-invasive electroencephalogram device, an electroencephalogram is generated, and the electroencephalogram generates an emotion measurement through the electroencephalogram emotion measurement model, which is conducive to optimizing speech generation under emotion measurement results and synthesizing emotionally rich speech that meets the empathy requirements of the audiences.

### BRIEF DESCRIPTION OF THE DRAWINGS

To more clearly illustrate the technical solutions in the embodiments of the present invention or in the prior art, the drawings required to be used in the description of the embodiments or the prior art are briefly introduced below. It is obvious that the drawings in the description below are merely embodiments of the present invention, and those of ordinary skill in the art can obtain other drawings according to the drawings provided without creative efforts.

FIG. 1 is a flow chart of an intelligent synthesis method for Cantonese speech based on electroencephalogram emotion measurement according to the present invention;

FIG. 2 is a schematic diagram of electroencephalogram emotion data labeling according to the present invention;

FIG. 3 is a schematic diagram of six electroencephalogram emotion measurements according to the present invention;

FIG. 4 is a flow chart of noise removal pretreatment performed on emotion extremum group data according to the present invention;

FIG. 5 is a schematic diagram of a structure of an electroencephalogram emotion measurement model according to the present invention;

FIG. 6 is a schematic diagram of feature distribution according to the present invention;

FIG. 7 is a framework diagram of an emotional speech synthesis model according to the present invention; and

FIG. 8 is a flow chart of dubbing based on an electroencephalogram emotion measurement model and an emotional speech synthesis model provided by an embodiment of the present invention.

### DETAILED DESCRIPTION OF THE EMBODIMENTS

The following clearly and completely describes the technical solutions in the embodiments of the present invention with reference to drawings in the embodiments of the present invention. It is clear that the described embodiments are merely a part rather than all of the embodiments of the present invention. Based on the examples of the present invention, all other examples obtained by those of ordinary skill in the art without creative efforts shall fall within the protection scope of the present invention.

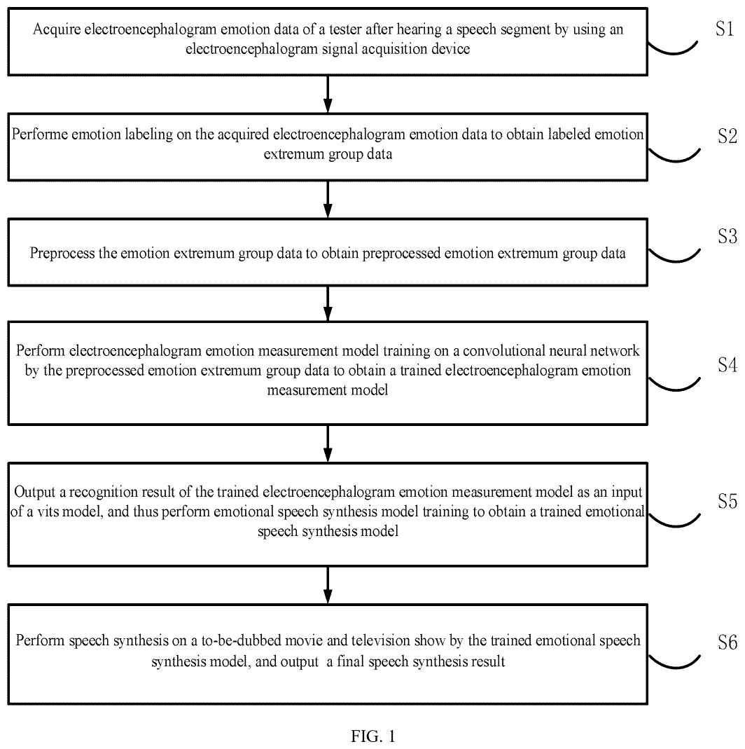

Referring to FIG. 1, the present invention discloses an intelligent synthesis method for Cantonese speech based on electroencephalogram emotion measurement, which includes the following steps:
S1. data acquisition: acquiring electroencephalogram emotion data of a tester after hearing a speech segment by using an electroencephalogram signal acquisition device;
S2. data labeling: performing emotion labeling on the acquired electroencephalogram emotion data to obtain labeled emotion extremum group data;
S3. data preprocess: preprocessing the emotion extremum group data to obtain preprocessed emotion extremum group data;
S4. electroencephalogram emotion measurement model training: performing electroencephalogram emotion measurement model training on a convolutional neural network by the preprocessed emotion extremum group data to obtain a trained electroencephalogram emotion measurement model;
S5. emotional speech synthesis model training: outputting a recognition result of the trained electroencephalogram emotion measurement model as an input of a vits model, and thus performing emotional speech synthesis model training to obtain a trained emotional speech synthesis model; and
S6. speech synthesis: performing speech synthesis on a to-be-dubbed movie and television show by the trained emotional speech synthesis model, and outputting a final speech synthesis result.

Further, the acquiring electroencephalogram emotion data of a tester after hearing a speech segment by using an electroencephalogram signal acquisition device in the S1 is specifically as follows:
a tester wears an electroencephalogram acquisition helmet and calibrates positions of electrodes, a recorder and a data acquisition software are started in sequence, the tester completes calibration processes of opening eyes, closing eyes and clicking a mouse according to instructions of the data acquisition software, a computer calculates signal calibration time offset in the calibration process, the tester hears speech segments, and the electroencephalogram acquisition helmet acquires electroencephalogram data of the tester, and marks time for starting playing and ending playing of the speech segments in the electroencephalogram data.

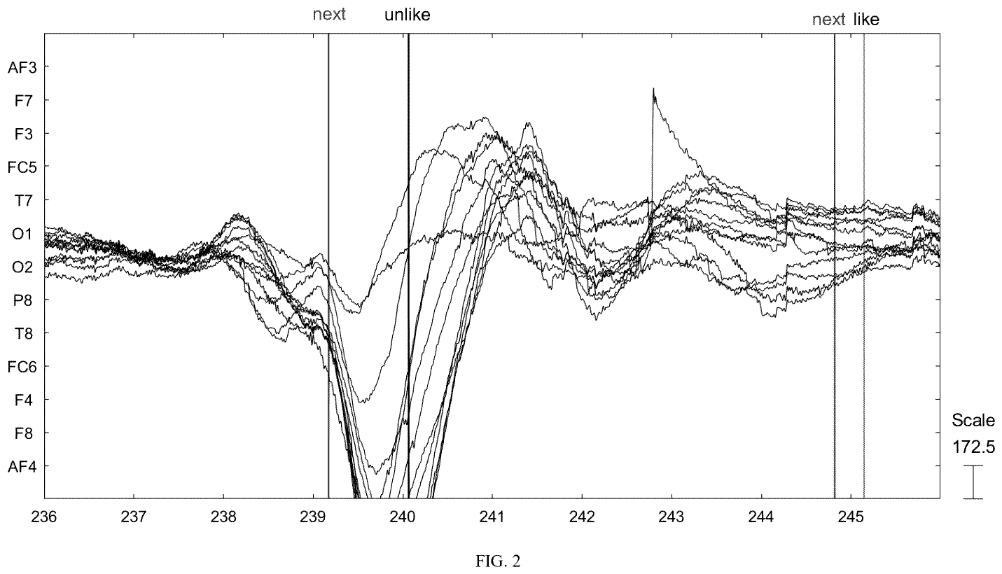

Specifically, physiological saline is added to electrodes of a multi-point electroencephalogram device, and the electrodes are installed. A tester wears an electroencephalogram acquisition helmet, and calibrates positions of the electrodes. A recorder and a data acquisition software are started in sequence to proceed a software calibration stage. The tester completes calibration processes of opening eyes, closing eyes and clicking a mouse according to instructions of the software, and a computer calculates signal calibration time offset T_{offset} in the calibration process. The tester then listens to emotional speech segments. This process acquires electroencephalogram data and marks the data with timestamps T_{begin} [symbol] T_{end} of when the speech segments begin and end. Finally, the electroencephalogram data is labeled with the calculated signal calibration time offset T_{offset}, and a time index of the electroencephalogram data segment is T_{begin}+T_{offset}:T_{end}+T_{offset}. A schematic diagram of human electroencephalogram data timestamp labeling is shown in FIG. 2. The electroencephalogram and corresponding speech labeling samples are (Sᵢ, Xᵢ, c), where Sᵢ is a i^{th} electroencephalogram sample, Xᵢ is a i^{th} audio sample, and the emotion category is c.

Further, the performing emotion labeling on the acquired electroencephalogram emotion data to obtain labeled emotion extremum group data in the S2 is specifically as follows:
the electroencephalogram emotion data obtained in the S1 are subjected to feature processing, electroencephalogram emotion data capable of representing emotion extrema are screened out, and emotion polarity labeling is performed on the electroencephalogram emotion data.

Further, the emotion extremum group data are an extremum group a, an extremum group b and an extremum group c classifying six emotions according to bipolarity of emotions.

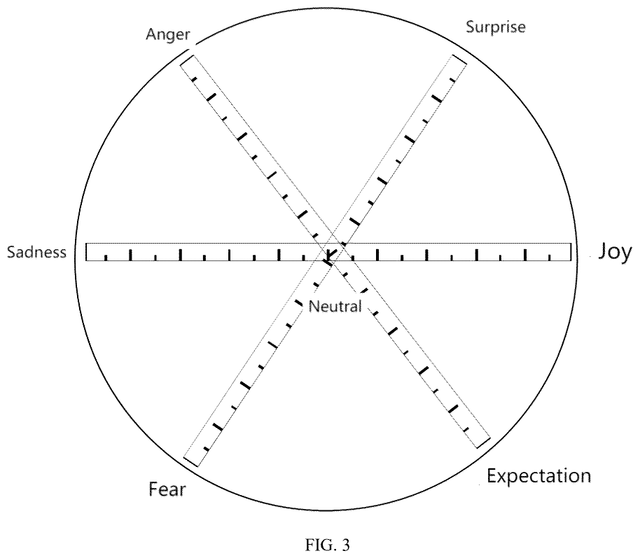

Specifically, the six basic emotions are classified according to bipolarity of emotions, including three groups of extrema: [sadness, joy], [anger, expectation], and [fear, surprise]. Each group is called an emotion measurement scale, a central intersection of which represents neutral emotion, and continuous values between the two extrema represent the intensity of the emotion in this group, as shown in FIG. 3. The sample is represented as (Sᵢ, Xᵢ, e_{y}), where S represents electroencephalogram data, X represents speech data, e represents emotion measurement value, y∈{a, b, c}, a represents an extremum group of [sadness, joy], b represents an extremum group of [anger, expectation], c represents an extremum group of [fear, surprise], e∈[0,1], and e is generally 1 or 0 when labeled.

Further, noise removal pretreatment is performed on the emotion extremum group data in the S3 to obtain emotion extremum group data after noise removal.

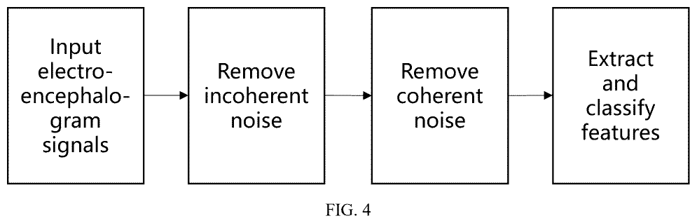

Specifically, more noise is inevitably introduced into the electroencephalogram data acquired by the safer non-invasive electroencephalogram acquisition device, so that the noise is mainly removed in the process of analyzing the electroencephalogram, and the noise includes incoherent noise and coherent noise. The incoherent noise refers to a noise having a frequency feature greatly different from that of a useful signal, and such noise is reflected as additive noise on a frequency domain and is easily removed. While the coherent noise refers to a noise having a frequency feature similar to those of a desired signal, such noise is easily mixed therein and is not easily removed. The noise removal process is shown in FIG. 4.

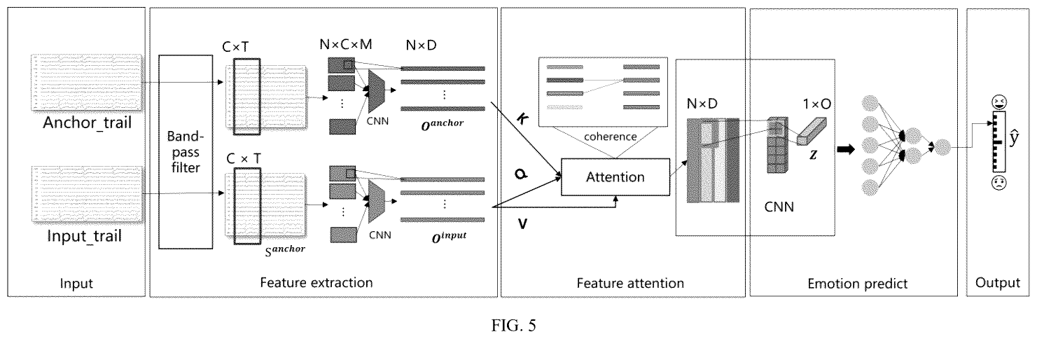

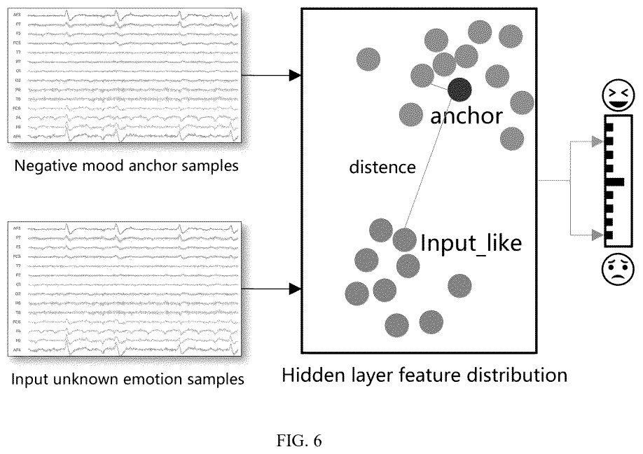

Further, as shown in FIG. 5, the performing electroencephalogram emotion measurement model training on a convolutional neural network by the preprocessed emotion extremum group data to obtain a trained electroencephalogram emotion measurement model in the S4 is specifically as follows:
S41. inputting a signal: the input signal has two sections, including anchor sample data anchor and real-time input electroencephalogram data input, and the anchor sample data is the emotion extremum group data obtained in S3 after noise removal;
S42. extracting a feature: the electroencephalogram emotion measurement model mainly captures a time period of emotion fluctuations, so that the emotion extremum group data after noise removal is framed by a window sliding method, and framed data is represented as follows:

$${S}^{input} = {S}_{1}^{input} ⊕ {S}_{2}^{input} ⊕ {S}_{3}^{i nput} ⊕ … ⊕ {S}_{N}^{input} , {S}_{n}^{i nput} ∈ {ℝ}^{C \times M} ;$$

$${S}^{a n c h o r} = {S}_{1}^{a n c h o r} ⊕ {S}_{2}^{a n c h o r} ⊕ {S}_{3}^{a n c h o r} ⊕ … ⊕ {S}_{N}^{a n chor} , {S}_{n}^{a n c h o r} ∈ {ℝ}^{C \times M}$$

wherein S^{anchor}∈ [symbol] ^{C×T}, S^{input}∈ [symbol] ^{C×T}, and C are a number of channels, T is a time length, [symbol] is a real number field, M is a window size, ⊕ is splicing operation, and N is a number of frames;
as shown in FIG. 6, the feature extraction performed on each frame by the convolutional neural network is represented as follows:

$${O}_{n} ( i , j ) = {\sum }_{c} {\sum }_{m} {S}_{n} ( i + c , j + m ) K ( c , m )$$

wherein K is a two-dimensional convolution kernel, and the convolution is performed along a channel vertically and along a time axis horizontally, Sₙ is each frame of signal, Oₙ∈ [symbol] ^{1×D} is a feature map representing an output result of each frame of signal after convolution, D is a D-dimensional vector, i is a vertical position index of a feature map pixel, j is a horizontal position index of a feature map pixel, c is a vertical position index of the convolution kernel, and m is a horizontal position index of the convolution kernel;
S43. anchoring coherent noise removal module based on an attention mechanism: the attention mechanism is applied to eliminate features similar to the anchor data, features of the anchor sample data are taken as K, and features of the emotion extremum group data after noise removal are taken as Q and V, which are represented as:

$$K = [ {O}_{1}^{a n c h o r} ; {O}_{2}^{a n c h o r} ; {O}_{3}^{a n c h o r} ; … ; {O}_{N}^{a n c h o r} ] , K ∈ {ℝ}^{N \times D} ;$$

$$Q = V = [ {O}_{1}^{input} ; {O}_{2}^{input} ; {O}_{3}^{input} ; … ; {O}_{N}^{input} ] , Q , V ∈ {ℝ}^{N \times D} ;$$

a distance is represented as:

$$Distance ( K , Q ) = ( {d}_{ij} ) , {d}_{ij} = {‖ {O}_{i}^{a n c h o r} - {O}_{j}^{input} ‖}_{2} ;$$

a distance from the attention mechanism is represented as:

$${A}^{input} = Attention ( Q , K , V ) = softmax ( Distance ( K , Q ) ) V , {A}^{input} ∈ {ℝ}^{N \times D}$$

wherein dᵢⱼ is a distance between an i^{th} feature and a j^{th} feature;
S44. classifying by a classifier: after a feature of A^{input} is compressed by a multi-layer convolutional neural network, the compressed feature is input into a fully connected neural network for emotion measurement prediction:

$$Z = convolution ( {A}^{i nput} ) , Z ∈ {ℝ}^{O}$$

$$\hat{e} = sigmod ( ANN ( Z ) ) , \hat{e} ∈ [ 0 , 1 ]$$

wherein Z is a flattened feature vector after convolution operation, a vector dimension is O, ANN is a fully connected neural network function, ê is a scalar value of 0-1 representing emotion measurement, the closer a predicted value is to 0, the more similar an input electroencephalogram signal input_trail is to an electroencephalogram signal anchor_trail for anchoring, otherwise, the more dissimilar, and an emotion measurement value of the input electroencephalogram signal is judged according to the scalar value; and
S45. training each sample labeled with the emotion measurement scale to obtain three trained electroencephalogram emotion measurement models

$$\{ {M}_{a}^{e e g} , {M}_{b}^{e e g} , {M}_{c}^{e e g} \} .$$

Specifically, the emotion extremum group data after the noise removal in the S42 feature extraction includes a plurality of frequency components, and the emotion is most influenced by the electroencephalogram in a frequency range from 0 Hertz (Hz) to 64 Hz according to the research, so that most incoherent background noise, such as electric signals generated by eye movement and muscle movement, may be removed after the emotion extremum group data with noise removed is filtered.

Further, the outputting a recognition result of the trained electroencephalogram emotion measurement model as an input of a vits model and thus performing emotional speech synthesis model training to obtain a trained emotional speech synthesis model in the S5 is specifically as follows:
the emotional speech synthesis model is divided into model learning speech reconstruction training and model emotion enhancement learning training;
S51. model learning speech reconstruction: performing model training by using the preprocessed emotion extremum group data obtained in the S3, learning speech reconstruction, and embedding emotion information in a text encoder in a vector form; wherein a loss function is a difference between a generated speech Mel spectrogram \hat{X}ₘₑₗ and a Mel spectrogram Xₘₑₗ of a real sample, that is, whether the speech is reconstructed, and a loss value for each sample is represented as:

$${loss}_{stage 1} = {loss}_{recon} = {‖ {X}_{mel} - {\hat{X}}_{mel} ‖}_{1} ;$$

S52. model emotion enhancement learning: performing emotion recognition on Cantonese speech generated by artificial intelligence (AI) using the trained electroencephalogram emotion measurement model obtained in the S4, importing a feedback signal of a dubber on an AI dubbing emotion by a device to calibrate a coding loss function of an AI dubbing emotion coder, calculating a loss value, and completing model reinforcement learning training for the emotional speech synthesis model, wherein a total loss value calculation formula is represented as:

$${loss}_{stage 2} = {αloss}_{recon} + {βloss}_{emotion}$$

$${loss}_{emotion} = - ( e log ( \hat{e} ) + ( 1 - e ) log ( 1 - \hat{e} ) )$$

wherein α and β are weight coefficients of reconstruction loss and emotion loss in a total loss of the model reinforcement learning.

Specifically, the S52 model emotion enhancement learning is specifically as follows: a tester (a professional dubber) wears an electroencephalogram acquisition device, evaluates speeches generated by hearing the model and performs emotion selection operation, selects among {sadness, joy, anger, expectation, fear, surprise}, and selects emotion categories and measurement l. When the tester is making a selection, the system automatically records a timestamp T_{begin} of the operation, then calculates a correction value T_{offset}, intercepts an electroencephalogram signal as an input of the electroencephalogram emotion measurement model

$${M}_{l}^{eeg}$$

by using the corrected label timestamp T_{label}, and finally obtains ê output by the model; and then calculates the loss value to perform secondary training on the Cantonese speech generation model.

The loss function includes an error between a model generated speech and a real speech, and an error between an AI dubbing emotion measurement of a professional dubber monitored by an electroencephalograph device and a real emotion label. Here, the cross entropy form is used. The loss value for each sample may be represented as:

$${loss}_{stage 2} = {αloss}_{recon} + {βloss}_{emotion}$$

$${loss}_{emotion} = - ( e log ( \hat{e} ) + ( 1 - e ) log ( 1 - \hat{e} ) )$$

α and β are hyper-parameters, and are represented as a weight coefficient of reconstruction loss and emotion loss in the total loss of the model reinforcement learning.

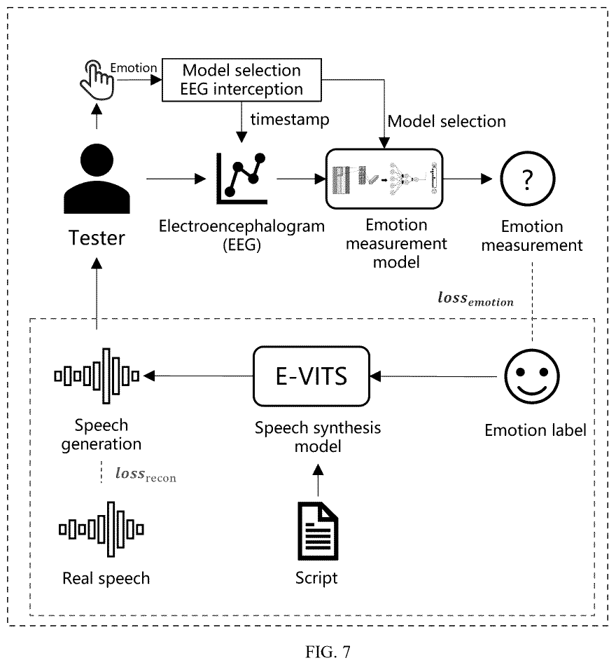

Specifically, the basic framework of the emotional speech synthesis model for Cantonese speech formed by the present invention is vits, which is an end-to-end speech synthesis model of an automatic encoder based on variation of the confrontation learning condition. The speech emotion information is embedded in a text encoder of the VIT model. The speech emotion information may come from artificial labeling or electroencephalogram emotion recognition. The emotional speech synthesis model is divided into model learning speech reconstruction training and model emotion enhancement learning training, and the training process is shown in FIG. 7.

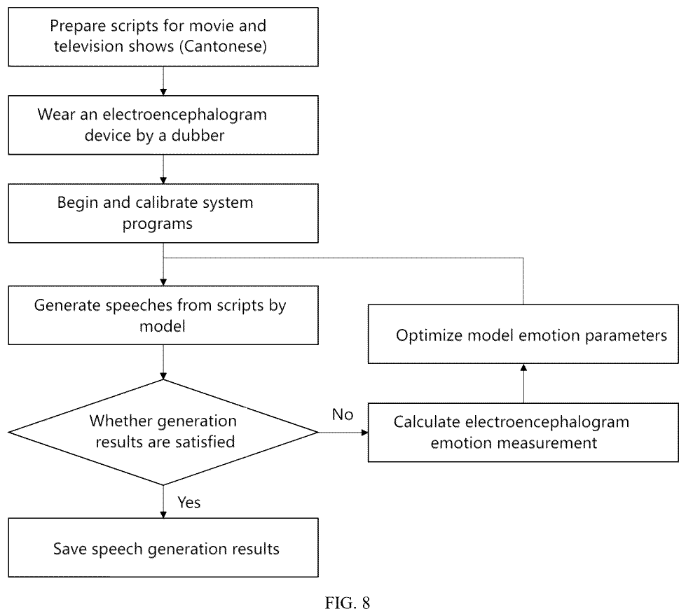

In a specific embodiment, to solve the problem of insufficient emotional expression in traditional Cantonese speech synthesis for movie and television shows trained by explicit text emotion labels, an intelligent synthesis method for Cantonese speech for movie and television shows based on electroencephalogram emotion measurement is proposed, as shown in FIG. 8. The specific method is as follows: preparing a Cantonese speech script according to a movie and television show content, then wearing an electroencephalogram device by a dubber; beginning a dubbing program and performing time calibration; inputting a text content into an emotional speech synthesis model; evaluating a model generation effect by the dubber; if satisfied with the generation effect, saving a speech generation result; if dissatisfied, reselecting the emotion by the dubber, automatically electroencephalogram obtaining data and inputting an electroencephalogram emotion measurement model by a system to calculate the measurement to optimize the emotional speech synthesis model, and then regenerating the speech, continuing to evaluate by the dubber until the generation effect is satisfied, then ending the speech generation and saving the speech generation result.

Corresponding to the method described in FIG. 1, an embodiment of the present invention further provides an intelligent synthesis system for Cantonese speech based on electroencephalogram emotion measurement, which is configured to specifically implement the method in FIG. 1, and specifically includes: a data acquisition module, a data labeling module, a data preprocessing module, an electroencephalogram emotion measurement model training module, an emotional speech synthesis model training module, and a speech synthesis module;
the data acquisition module is connected to an input end of the data labeling module, and is configured to acquire electroencephalogram emotion data of a tester after hearing a speech segment by using an electroencephalogram signal acquisition device;
the data labeling module is connected to an input end of the data preprocessing module, and is configured to perform emotion labeling on the acquired electroencephalogram emotion data to obtain labeled emotion extremum group data;
the data preprocessing module is connected to an input end of the electroencephalogram emotion measurement model training module, and is configured to preprocess the emotion extremum group data to obtain preprocessed emotion extremum group data;
the electroencephalogram emotion measurement model training module is connected to an input end of the emotional speech synthesis model training module, and is configured to perform electroencephalogram emotion measurement model training on a convolutional neural network by the preprocessed emotion extremum group data to obtain a trained electroencephalogram emotion measurement model;
the emotional speech synthesis model training module is connected to an input end of the speech synthesis module, and is configured to output a recognition result of the trained electroencephalogram emotion measurement model as an input of a vits model and thus perform emotional speech synthesis model training to obtain a trained emotional speech synthesis model; and
the speech synthesis module is configured to perform speech synthesis on a to-be-dubbed movie and television show by the trained emotional speech synthesis model and output a final speech synthesis result.

The embodiments in the specification are all described in a progressive manner, and each embodiment focuses on differences from other embodiments, and portions that are the same and similar between the embodiments may be referred to each other. Since the apparatus disclosed in the embodiment corresponds to the method disclosed in the embodiment, the description is relatively simple, and reference may be made to the partial description of the method.

The above description of the disclosed embodiments enables those skilled in the art to implement or use the present invention. Various modifications to these embodiments will be readily apparent to those skilled in the art, and the generic principles defined herein may be applied to other embodiments without departing from the spirit or scope of the present invention. Thus, the present invention is not intended to be limited to these embodiments shown herein but is to be accorded the broadest scope consistent with the principles and novel features disclosed herein.

1. An intelligent synthesis method for Cantonese speech based on electroencephalogram emotion measurement, comprising the following steps:
S1. data acquisition: acquiring electroencephalogram emotion data of a tester after hearing a speech segment by using an electroencephalogram signal acquisition device;
S2. data labeling: performing emotion labeling on the electroencephalogram emotion data to obtain labeled emotion extremum group data;
S3. data preprocess: preprocessing the labeled emotion extremum group data to obtain preprocessed emotion extremum group data;
S4. electroencephalogram emotion measurement model training: performing electroencephalogram emotion measurement model training on a convolutional neural network by the preprocessed emotion extremum group data to obtain a trained electroencephalogram emotion measurement model;
S5. emotional speech synthesis model training: outputting a recognition result of the trained electroencephalogram emotion measurement model as an input of a vits model, and thus performing emotional speech synthesis model training to obtain a trained emotional speech synthesis model; and
S6. speech synthesis: performing speech synthesis on a to-be-dubbed movie and television show by the trained emotional speech synthesis model, and outputting a final speech synthesis result;
wherein the step of performing electroencephalogram emotion measurement model training on the convolutional neural network by the preprocessed emotion extremum group data to obtain the trained electroencephalogram emotion measurement model in the S4 comprises:
S41. inputting a signal: the input signal has two sections, comprising anchor sample data anchor and real-time input electroencephalogram data input, and the anchor sample data is the preprocessed emotion extremum group data obtained in S3 after noise removal;
S42. extracting a feature: the electroencephalogram emotion measurement model captures a time period of emotion fluctuations, so that the preprocessed emotion extremum group data after noise removal is framed by a window sliding method, and framed data is represented as follows:
$${S}^{input} = {S}_{1}^{input} ⊕ {S}_{2}^{input} ⊕ {S}_{3}^{input} ⊕ ... ⊕ {S}_{N}^{input} , {S}_{n}^{input} ∈ {ℝ}^{C \times M} ;$$
$${S}^{anchor} = {S}_{1}^{anchor} ⊕ {S}_{2}^{anchor} ⊕ {S}_{3}^{anchor} ⊕ ... ⊕ {S}_{N}^{anchor} , {S}_{n}^{anchor} ∈ {ℝ}^{C \times M}$$
wherein S^{anchor}∈ [symbol] ^{C×T}, S^{input}∈ [symbol] ^{C×T}, and C are a number of channels, T is a time length, [symbol] is a real number field, M is a window size, ⊕ is splicing operation, and N is a number of frames;
feature extraction performed on each frame by the convolutional neural network is represented as follows:
$${O}_{n} ( i , j ) = {\sum }_{c} {\sum }_{m} {S}_{n} ( i + c , j + m ) K ( c , m )$$
wherein K is a two-dimensional convolution kernel, convolution is performed along a channel vertically and along a time axis horizontally, Sₙ is each frame of signal, Oₙ∈ [symbol] ^{1×D} is a feature map representing an output result of each frame of signal after convolution, D is a D-dimensional vector, i is a vertical position index of a feature map pixel, j is a horizontal position index of the feature map pixel, c is a vertical position index of the two-dimensional convolution kernel, and m is a horizontal position index of the two-dimensional convolution kernel;
S43. anchoring coherent noise removal module based on an attention mechanism: the attention mechanism is applied to eliminate features similar to the anchor sample data, features of the anchor sample data are taken as K, and features of the real-time input electroencephalogram data are taken as Q and V represented as:
$$K = [ {O}_{1}^{anchor} ; {O}_{2}^{anchor} ; {O}_{3}^{anchor} ; ... ; {O}_{N}^{anchor} ] , K ∈ {ℝ}^{N \times D} ;$$
$$Q = V = [ {O}_{1}^{input} ; {O}_{2}^{input} ; {O}_{3}^{input} ; ... ; {O}_{N}^{input} ] ; Q , V ∈ {ℝ}^{N \times D} ;$$
a distance is represented as:
$$Distance ( K , Q ) = ( {d}_{ij} ) , {d}_{ij} = {‖ {O}_{i}^{anchor} - {O}_{j}^{input} ‖}_{2} ;$$
a distance from the attention mechanism is represented as:
$${A}^{input} = Attention ( Q , K , V ) = soft max ( Distance ( K , Q ) ) V , {A}^{input} ∈ {ℝ}^{N \times D}$$
wherein dᵢⱼ is a distance between an i^{th} feature and a j^{th} feature;
S44. classifying by a classifier: after a feature of A^{input} is compressed by a multi-layer convolutional neural network to obtain a compressed feature, the compressed feature is input into a fully connected neural network for emotion measurement prediction:
$$Z = convolution ( {A}^{input} ) , Z ∈ {ℝ}^{O}$$
$$\hat{e} = sig mod ( ANN ( Z ) ) , \hat{e} ∈ [ 0 , 1 ]$$
wherein Z is a flattened feature vector after convolution operation, a vector dimension is O, ANN is a fully connected neural network function, ê is a scalar value of 0-1 representing emotion measurement, the closer a predicted value is to 0, the more similar an input electroencephalogram signal input_trail is to an electroencephalogram signal anchor_trail for anchoring, otherwise, the more dissimilar, and an emotion measurement value of the input electroencephalogram signal is judged according to the scalar value; and
S45. training each sample labeled with an emotion measurement scale to obtain three trained electroencephalogram emotion measurement models
$$\{ {M}_{a}^{eeg} , {M}_{b}^{eeg} , {M}_{c}^{eeg} \} .$$

2. The intelligent synthesis method for Cantonese speech based on electroencephalogram emotion measurement according to claim 1, wherein
the step of acquiring the electroencephalogram emotion data of the tester after hearing the speech segment by using the electroencephalogram signal acquisition device in the S1 is implemented as follows:
the tester wears an electroencephalogram acquisition helmet and calibrates positions of electrodes, a recorder and a data acquisition software are started in sequence, the tester completes calibration processes of opening eyes, closing eyes and clicking a mouse according to instructions of the data acquisition software, a computer calculates signal calibration time offset in a calibration process, the tester hears speech segments, and the electroencephalogram acquisition helmet acquires electroencephalogram data of the tester, and marks time for starting playing and ending playing of the speech segments in the electroencephalogram data.

3. The intelligent synthesis method for Cantonese speech based on electroencephalogram emotion measurement according to claim 1, wherein
the step of performing emotion labeling on the electroencephalogram emotion data to obtain the labeled emotion extremum group data in the S2 is implemented as follows:
the electroencephalogram emotion data obtained in the S1 are subjected to feature processing, electroencephalogram emotion data for representing emotion extrema are screened out, and emotion polarity labeling is performed on the electroencephalogram emotion data.

4. The intelligent synthesis method for Cantonese speech based on electroencephalogram emotion measurement according to claim 3, wherein
the labeled emotion extremum group data are an extremum group a, an extremum group b and an extremum group c classifying six emotions according to bipolarity of emotions.

5. The intelligent synthesis method for Cantonese speech based on electroencephalogram emotion measurement according to claim 1, wherein
noise removal pretreatment is performed on the labeled emotion extremum group data in the S3 to obtain the preprocessed emotion extremum group data after noise removal.

6. The intelligent synthesis method for Cantonese speech based on electroencephalogram emotion measurement according to claim 1, wherein
the step of outputting the recognition result of the trained electroencephalogram emotion measurement model as the input of the vits model and thus performing emotional speech synthesis model training to obtain the trained emotional speech synthesis model in the S5 is implemented as follows:
the emotional speech synthesis model is divided into model learning speech reconstruction training and model emotion enhancement learning training;
S51. model learning speech reconstruction: performing model training by using the preprocessed emotion extremum group data obtained in the S3, learning speech reconstruction, and embedding emotion information in a text encoder in a vector form; wherein a loss function is a difference between a generated speech Mel spectrogram \hat{X}ₘₑₗ and a Mel spectrogram Xₘₑₗ of a real sample, that is, whether the speech is reconstructed, and a loss value of a reconstruction loss for each sample is represented
$${loss}_{stage 1} = {loss}_{recon} = {‖ {X}_{mel} - {\hat{X}}_{mel} ‖}_{1} ;$$
S52. model emotion enhancement learning: performing emotion recognition on Cantonese speech generated by artificial intelligence (AI) using the trained electroencephalogram emotion measurement model obtained in the S4, importing a feedback signal of a dubber on an AI dubbing emotion by a device to calibrate a coding loss function of an AI dubbing emotion coder, calculating a loss value of an emotion loss, and completing model reinforcement learning training for the emotional speech synthesis model, wherein a total loss value calculation formula is represented as:
$${loss}_{stage 2} = {αloss}_{recon} + {βloss}_{emotion}$$
$${loss}_{emotion} = - ( e log ( \hat{e} ) + ( 1 - e ) log ( 1 - \hat{e} ) )$$
wherein α and β are weight coefficients of the reconstruction loss and the emotion loss in a total loss of the model reinforcement learning training.

7. An intelligent synthesis system for Cantonese speech based on electroencephalogram emotion measurement, applied to the intelligent synthesis method for Cantonese speech based on electroencephalogram emotion measurement according to claim 1, and comprising: a data acquisition module, a data labeling module, a data preprocessing module, an electroencephalogram emotion measurement model training module, an emotional speech synthesis model training module, and a speech synthesis module; wherein
the data acquisition module is connected to an input end of the data labeling module, and is configured to acquire electroencephalogram emotion data of a tester after hearing a speech segment by using an electroencephalogram signal acquisition device;
the data labeling module is connected to an input end of the data preprocessing module, and is configured to perform emotion labeling on the electroencephalogram emotion data to obtain labeled emotion extremum group data;
the data preprocessing module is connected to an input end of the electroencephalogram emotion measurement model training module, and is configured to preprocess the labeled emotion extremum group data to obtain preprocessed emotion extremum group data;
the electroencephalogram emotion measurement model training module is connected to an input end of the emotional speech synthesis model training module, and is configured to perform electroencephalogram emotion measurement model training on a convolutional neural network by the preprocessed emotion extremum group data to obtain a trained electroencephalogram emotion measurement model;
the emotional speech synthesis model training module is connected to an input end of the speech synthesis module, and is configured to output a recognition result of the trained electroencephalogram emotion measurement model as an input of a vits model and thus perform emotional speech synthesis model training to obtain a trained emotional speech synthesis model; and
the speech synthesis module is configured to perform speech synthesis on a to-be-dubbed movie and television show by the trained emotional speech synthesis model and output a final speech synthesis result.

8. The intelligent synthesis system for Cantonese speech based on electroencephalogram emotion measurement according to claim 7, wherein in the intelligent synthesis method for Cantonese speech based on electroencephalogram emotion measurement,
the step of acquiring the electroencephalogram emotion data of the tester after hearing the speech segment by using the electroencephalogram signal acquisition device in the S1 is implemented as follows:
the tester wears an electroencephalogram acquisition helmet and calibrates positions of electrodes, a recorder and a data acquisition software are started in sequence, the tester completes calibration processes of opening eyes, closing eyes and clicking a mouse according to instructions of the data acquisition software, a computer calculates signal calibration time offset in a calibration process, the tester hears speech segments, and the electroencephalogram acquisition helmet acquires electroencephalogram data of the tester, and marks time for starting playing and ending playing of the speech segments in the electroencephalogram data.

9. The intelligent synthesis system for Cantonese speech based on electroencephalogram emotion measurement according to claim 7, wherein in the intelligent synthesis method for Cantonese speech based on electroencephalogram emotion measurement,
the step of performing emotion labeling on the electroencephalogram emotion data to obtain the labeled emotion extremum group data in the S2 is implemented as follows:
the electroencephalogram emotion data obtained in the S1 are subjected to feature processing, electroencephalogram emotion data for representing emotion extrema are screened out, and emotion polarity labeling is performed on the electroencephalogram emotion data.

10. The intelligent synthesis system for Cantonese speech based on electroencephalogram emotion measurement according to claim 9, wherein in the intelligent synthesis method for Cantonese speech based on electroencephalogram emotion measurement,
the labeled emotion extremum group data are an extremum group a, an extremum group b and an extremum group c classifying six emotions according to bipolarity of emotions.

11. The intelligent synthesis system for Cantonese speech based on electroencephalogram emotion measurement according to claim 7, wherein in the intelligent synthesis method for Cantonese speech based on electroencephalogram emotion measurement,
noise removal pretreatment is performed on the labeled emotion extremum group data in the S3 to obtain the preprocessed emotion extremum group data after noise removal.

12. The intelligent synthesis system for Cantonese speech based on electroencephalogram emotion measurement according to claim 7, wherein in the intelligent synthesis method for Cantonese speech based on electroencephalogram emotion measurement,
the step of outputting the recognition result of the trained electroencephalogram emotion measurement model as the input of the vits model and thus performing emotional speech synthesis model training to obtain the trained emotional speech synthesis model in the S5 is implemented as follows:
the emotional speech synthesis model is divided into model learning speech reconstruction training and model emotion enhancement learning training;
S51. model learning speech reconstruction: performing model training by using the preprocessed emotion extremum group data obtained in the S3, learning speech reconstruction, and embedding emotion information in a text encoder in a vector form; wherein a loss function is a difference between a generated speech Mel spectrogram \hat{X}ₘₑₗ and a Mel spectrogram Xₘₑₗ of a real sample, that is, whether the speech is reconstructed, and a loss value of a reconstruction loss for each sample is represented as:
$${loss}_{stage 1} = {loss}_{recon} = {‖ {X}_{mel} - {\hat{X}}_{mel} ‖}_{1} ;$$
S52. model emotion enhancement learning: performing emotion recognition on Cantonese speech generated by artificial intelligence (AI) using the trained electroencephalogram emotion measurement model obtained in the S4, importing a feedback signal of a dubber on an AI dubbing emotion by a device to calibrate a coding loss function of an AI dubbing emotion coder, calculating a loss value of an emotion loss, and completing model reinforcement learning training for the emotional speech synthesis model, wherein a total loss value calculation formula is represented as:
$${loss}_{stage 2} = {αloss}_{recon} + {βloss}_{emotion}$$
$${loss}_{emotion} = - ( e log ( \hat{e} ) + ( 1 - e ) log ( 1 - \hat{e} ) )$$
wherein α and β are weight coefficients of the reconstruction loss and the emotion loss in a total loss of the model reinforcement learning training.
